(19) United States
(12) Patent Application Publication
(10) Pub. No.: US 2012/0022274 A1
(43) Pub. Date: Jan. 26, 2012

(54) Crystalline Form of Orlistat and a Process Thereof

(76) Inventors: Umesh Sannachikkanna, Karnataka (IN); Chandrashekar Aswathanarayanappa, Karnataka (IN); Pullela Venkata Srinivas, Karnataka (IN)

(21) Appl. No.: 13/145,100

(22) PCT Filed: Mar. 6, 2009
(86) PCT No.: PCT/IN09/00157

§ 371 Date: Jul. 18, 2011

(30) Foreign Application Priority Data
Jan. 22, 2009 (IN) 00138/CHE/2009

(51) Int. Cl. C07D 305/12

(52) U.S. Cl. 549/328

(57) ABSTRACT

The present disclosure provides a crystalline form of orlistat, particularly form A of orlistat. The characterization of said form is performed using X-ray diffraction studies, IR and Differential Scanning calorimetry melting endotherm studies. Also, the disclosure provides a simple process to arrive at said crystalline form.

### FIELD OF THE INVENTION

The present invention is in relation to a new polymorph of Orlistat (Form A) and a process for the preparation thereof.

### BACKGROUND AND PRIOR ART OF THE INVENTION

The present invention relates to the new polymorph and process for the preparation of Orlistat which is known by chemical name N-Formyl-L-leucine (1S)-1-[[(2S,3S)-3-hexyl-4-oxo-2-oxetanyl]methyl]dodecyl ester which inhibits pancreas lipase and used in the control obesity and hyperlipidemia.

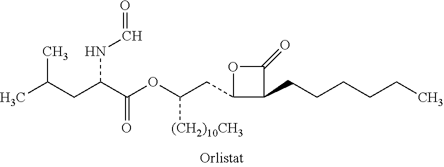

U.S. Pat. No. 4,598,089 discloses the preparation of Orlistat through fermentation. This patent also disclosed the purification of Orlistat by repeated chromatography which results tedious and costly.

Additionally, few international Patent Applications have disclosed crystalline forms of Orlistat, as well as processes for preparing the same. That includes U.S. Pat. No. 6,734,314 claims Form I and Form II of Orlistat and preparation thereof.

The present invention related to preparation of Orlistat with a different solid state property, in which new crystalline form.

The properties of chemical compounds are affected by many physical parameters. The crystallinity of a chemical compound is important with respect to formulation as a pharmaceutical which affects the flowability during processing and storage stability. Another important property of a pharmaceutical compound that may depend on crystallinity is its rate of dissolution. These practical physical characteristics are influenced by the conformation and orientation of molecules in the unit cell, which defines a particular crystalline form of a substance. Different crystalline forms may give rise to distinct spectroscopic properties that may be detectable by such analytical techniques as powder X-ray diffraction. A particular crystalline form may also give rise to thermal behavior. Thermal behavior is measured in the laboratory by such techniques as capillary melting point, thermogravimetric analysis (TGA), and differential scanning calorimetry (DSC), and can be used to distinguish some crystalline forms from others.

### BRIEF DESCRIPTION OF THE ACCOMPANYING DRAWINGS

FIG. 1 represents the XRD of crystalline solid Orlistat form A.

FIG. 2 represents the DSC of crystalline solid Orlistat form A.

FIG. 3 represents the IR of crystalline solid Orlistat form A.

### OBJECTIVES OF THE PRESENT INVENTION

The principle objective of the present invention is to provide a crystalline form of orlistat.

Another objective of the present invention is to provide novel polymorph of Orlistat and processes for preparation, which is very suitable for use on an industrial scale.

### STATEMENT OF THE INVENTION

Accordingly, the present invention is in relation to a crystalline form of Orlistat characterized by a XRD pattern with strong peaks at 4.0, 4.8, 5.4, 6.1, 9.0, 9.3, 9.8, 10.0, 10.8, 11.2, 12.2, 14.0, 15.0, 15.4, 15.8., 16.3, 16.9, 18.3, 18.6, 19.2, 19.6, 20.1, 20.5, 21.0, 21.4, 22.1, 22.6, 23.0, 23.6, 23.8, 24.6, 25.5, 26.1, 28.1, 29.3, 29.6, 29.9, 30.3, 31.9, 34.7, 40.0, 46.22±0.2 degrees 2θ.

### DETAILED DESCRIPTION OF THE INVENTION

The present invention is in relation to a crystalline form of Orlistat characterized by a

XRD pattern with strong peaks at 4.0, 4.8, 5.4, 6.1, 9.0, 9.3, 9.8, 10.0, 10.8, 11.2, 12.2, 14.0, 15.0, 15.4, 15.8, 16.3, 16.9, 18.3, 18.6, 19.2, 19.6, 20.1, 20.5, 21.0, 21.4, 22.1, 22.6, 23.0, 23.6, 23.8, 24.6, 25.5, 26.1, 28.1, 29.3, 29.6, 29.9, 30.3, 31.9, 34.7, 40.0, 46.22±0.2 degrees 2θ.

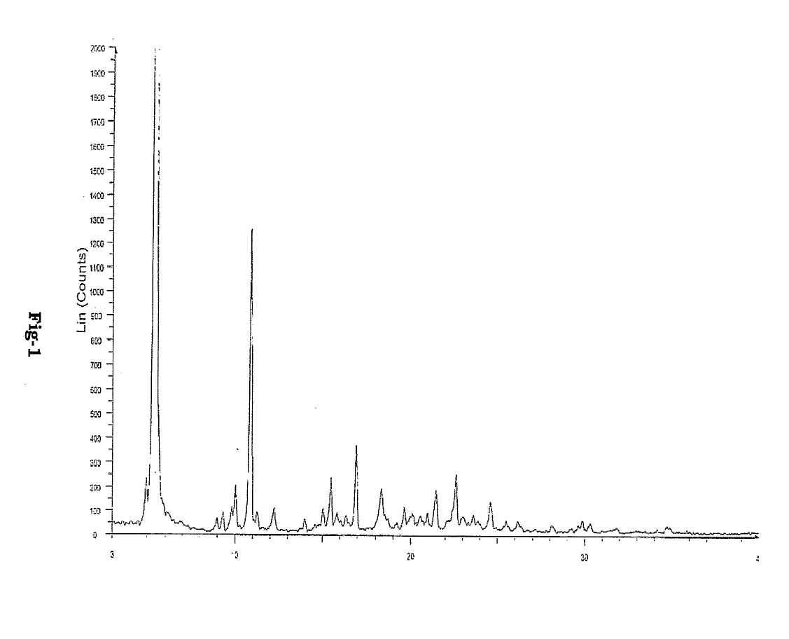

In another embodiment of the present invention the crystalline form is characterized by a XRD pattern substantially as shown in FIG. 1.

In yet another embodiment of the present invention the crystalline form is Orlistat Form A.

In still another embodiment of the present invention the crystalline form characterized by a DSC melting endotherm at 44.68° C.

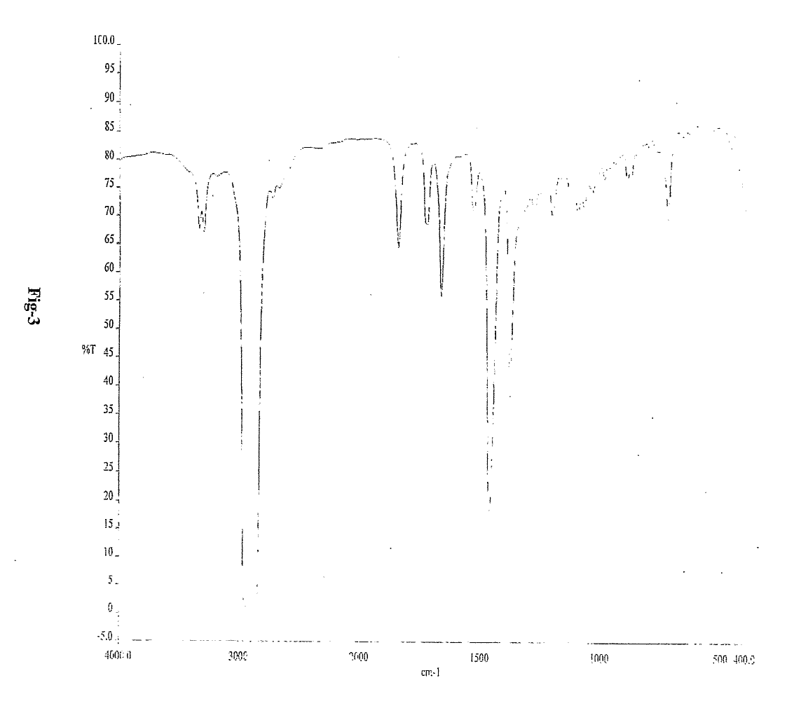

In still another embodiment of the present invention the crystalline form is characterized by IR substantially as shown in FIG. 3.

The present invention is in relation to a process for preparation of crystalline Orlistat Form A as recited in above embodiments, said process comprising steps of: addition of Orlistat in to a mixture of organic solvents; cooling the mixture to a lower temperature; isolation of crystalline solids; and drying.

In still another embodiment of the present invention said organic solvents are selecting from both polar and non-polar solvents.

In still another embodiment of the present invention the mixture of organic solvents is preferably mixture of acetone and heptane.

In still another embodiment of the present invention said lower temperature is less than 5° C.

In still another embodiment of the present invention said drying is vacuum tray drying.

The present invention provides the new crystalline form (Form A) of Orlistat.

The present crystalline solid Orlistat or hydrate or solvate thereof, characterized by XRD pattern, IR and a DSC melting endotherm.

The present invention provides a process of preparing pure form of Orlistat by crystallizing the solid Orlistat from a mixture of organic solvents. The organic solvents are selecting from both polar and non-polar solvents most preferably acetone and heptane mixture at the temperature less than 5° C.

The present invention provides a process of preparing crystalline solid orlistat, or hydrate or solvate thereof, characterized by data selected from the group consisting of a XRD pattern as depicted in FIG. 1, comprising the steps of:
a. addition of Orlistat in to a mixture of organic solvents,
b. cooling the mixture to a lower temperature,
c. isolation of crystalline solids and
d. drying.

The organic solvents are selected from both polar and non-polar solvents, preferably the mixture of acetone and heptane. Lower temperature is less than 5° C.

The present invention provides the new crystalline form (Form A) of Orlistat. This crystalline Orlistat is more stable and free flowing in nature. The purity of this compound is more than 99%. The crystallization method employed is able to remove both polar as well as non polar impurities.

The present crystalline solid Orlistat or hydrate or solvate thereof, characterized by XRD pattern, IR and a DSC melting endotherm.

The crystalline solid Orlistat is further characterized by a XRD pattern substantially as shown in FIG. 1.

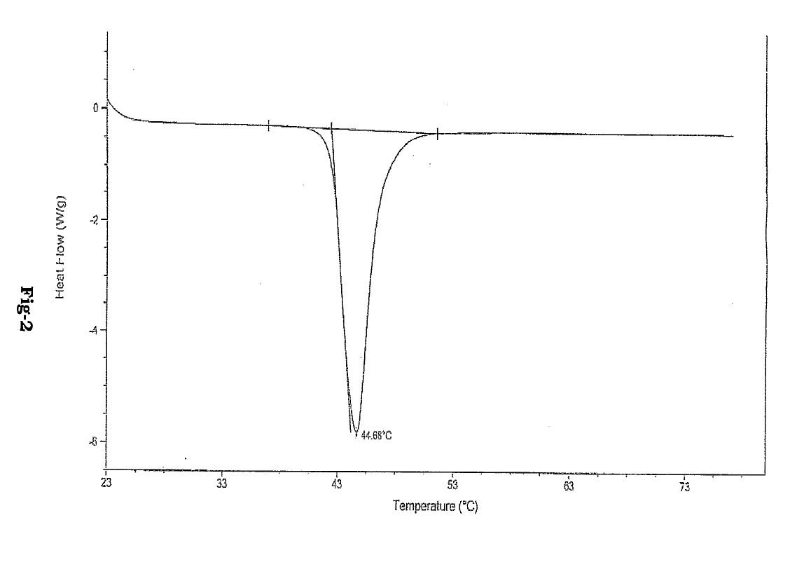

Preferably, the crystalline solid Orlistat characterized by a DSC melting endotherm at about 44.68° C. as shown in FIG. 2.

The present invention provides a process of preparing pure form of Orlistat by crystallizing the solid Orlistat from a mixture of organic solvents. The organic solvents are selecting from both polar and non-polar solvents most preferably acetone and heptane mixture at the temperature less than 5° C.

The present invention provides a process of preparing crystalline solid Orlistat, or hydrate or solvate thereof, characterized by data selected from the group consisting of a XRD pattern with peaks at 4.0, 4.8, 5.4, 6.1, 9.0, 9.3, 9.8, 10.0, 10.8, 11.2, 12.2, 14.0, 15.0, 15.4, 15.8, 16.3, 16.9, 18.3, 18.6, 19.2, 19.6, 20.1, 20.5, 21.0, 21.4, 22.1, 22.6, 23.0, 23.6, 23.8, 24.6, 25.5, 26.1, 28.1, 29.3, 29.6, 29.9, 30.3, 31.9, 34.7, 40.0, 46.22±0.2 degrees 2θ and a DSC melting endotherm at about 44.68° C., comprising the steps of:
a. addition of Orlistat in to a mixture of organic solvents,
b. cooling the mixture to a lower temperature,
c. isolation of crystalline solids and
d. drying.

The organic solvents are selected from both polar and non-polar solvents, preferably the mixture of acetone and heptane. Lower temperature is less than 5° C.

The present crystallization process can be repeated several times. The repetition of crystallization process will improve the quality of the crystalline Orlistat.

The technology of the instant Application is further elaborated with the help of following examples. However, the examples should not be construed to limit the scope of the invention.

### EXAMPLES

### Example: 1

10 g Orlistat was added in to a mixture of acetone-heptane and stirred to dissolve and cooled to 0 to 5° C., the mixture was stirred for four hours. Orlistat was crystallized slowly. Product was filtered and dried in vacuum tray dryer.

### Example: 2

250 g Orlistat was added in to a mixture of acetone-heptane and stirred to dissolve and cooled to 0 to 5° C., the mixture was stirred for four hours. Orlistat was crystallized slowly. Product was filtered and dried in vacuum tray dryer.

We claim:

1. A crystalline form of Orlistat characterized by a XRD pattern with strong peaks at 4.0, 4.8, 5.4, 6.1, 9.0, 9.3, 9.8, 10.0, 10.8, 11.2, 12.2, 14.0, 15.0, 15.4, 15.8, 16.3, 16.9, 18.3, 18.6, 19.2, 19.6, 20.1, 20.5, 21.0, 21.4, 22.1, 22.6, 23.0, 23.6, 23.8, 24.6, 25.5, 26.1, 28.1, 29.3, 29.6, 29.9, 30.3, 31.9, 34.7, 40.0, 46.22±0.2 degrees 2θ.

2. The crystalline form as claimed in claim 1, wherein the crystalline form is characterized by a XRD pattern substantially as shown in FIG. 1.

3. The crystalline form as claimed in claim 1, wherein the crystalline form is Orlistat Form A.

4. The crystalline form as claimed in claim 1, wherein the crystalline form characterized by a DSC melting endotherm at 44.68° C.

5. The crystalline form as claimed in claim 1, wherein the crystalline form is characterized by IR substantially as shown in FIG. 3.

6. A process for preparation of crystalline Orlistat Form A as claimed in claim 1, said process comprising steps of:
a) addition of Orlistat in to a mixture of organic solvents;
b) cooling the mixture to a lower temperature;
c) isolation of crystalline solids; and
d) drying.

7. The process as claimed in claim 6, wherein said organic solvents are selecting from both polar and non-polar solvents.

8. The process as claimed in claim 6, wherein the mixture of organic solvents is preferably mixture of acetone and heptane.

9. The process as claimed in claim 6, wherein said lower temperature is less than 5° C.

10. The process as claimed in claim 5, wherein said drying is vacuum tray drying.
